(19) United States
(12) Patent Application Publication
(10) Pub. No.: US 2025/0169795 A1
(43) Pub. Date: May 29, 2025

(54) ULTRASOUND DIAGNOSTIC APPARATUS

(71) Applicant: FUJIFILM Corporation, Tokyo (JP)

(72) Inventor: Yuki MITANI, Tokyo (JP)

(73) Assignee: FUJIFILM Corporation, Tokyo (JP)

(21) Appl. No.: 18/955,877

(22) Filed: Nov. 21, 2024

(30) Foreign Application Priority Data
Nov. 28, 2023 (JP) 2023-200581

(51) Int. Cl. A61B 8/08; A61B 8/00

(52) U.S. Cl. CPC A61B 8/485; A61B 8/463; A61B 8/54

(57) ABSTRACT

An ultrasound diagnostic apparatus including an information processing unit is provided. The information processing unit executes a B-mode image acquisition process of acquiring B-mode image data, an attenuation property acquisition process of acquiring attenuation property data, an attenuation distribution display process of displaying, on a display, an attenuation distribution image in which an attenuation property map indicated by the attenuation property data is superimposed on a B-mode image indicated by the B-mode image data, a setting process of setting an elastic property region of interest on a tomographic plane from which the B-mode image data has been acquired based on a user's operation, and an elastic property acquisition process is a process of acquiring elastic property data for the elastic property region of interest.

### CROSS-REFERENCE TO RELATED APPLICATION

This application claims priority under 35 USC 119 from Japanese Patent Application No. 2023-200581, filed 28 Nov. 2023, the disclosure of which is incorporated by reference herein.

### BACKGROUND OF THE INVENTION

### 1. Field of the Invention

The present disclosure relates to an ultrasound diagnostic apparatus, and more particularly to an apparatus for acquiring elastic property data and attenuation property data of a subject.

### 2. Description of the Related Art

Some ultrasound diagnostic apparatuses operate in an elasticity measurement mode that generates an elasticity distribution image or an attenuation measurement mode that generates an attenuation distribution image in addition to a B-mode that generates a B-mode image. Here, the elasticity distribution image is an image showing a distribution of elastic moduli, and the attenuation distribution image is an image showing a distribution of attenuation rates. The alignment of an ultrasound probe and the setting of a region of interest are performed using a B-mode image displayed on a display or the like, and the distribution of the elastic moduli or the distribution of the attenuation rates is measured for the set region of interest.

The following JP2010-99378A and JP6212160B disclose techniques for generating an elasticity distribution image. JP2020-138017A discloses a technique for generating a B-mode image, an elasticity distribution image, and an attenuation distribution image. JP2015-198843A discloses an ultrasound diagnostic apparatus that excites shear waves in a tissue of a subject by applying pulse vibrations to a probe in response to the movement of a user's hand.

### SUMMARY OF THE INVENTION

In general, in a case of generating an elasticity distribution image, an acoustic radiation force is applied to a biological tissue from an ultrasound probe, and shear waves for generating the elasticity distribution image are propagated through the biological tissue. This causes the ultrasound probe to heat up, so that a cooling period is provided after the ultrasonic wave is transmitted and before the next transmission is performed. By providing a cooling period, a sufficient frame rate may not be obtained in a case in which elasticity distribution images are repeatedly generated. Therefore, in a case in which a user performs setting operations such as aligning an ultrasound probe and setting a region of interest while checking an elasticity distribution image in addition to a B-mode image, the setting operations may be difficult.

An object of the present disclosure is to facilitate an operation for acquiring an ultrasound image such as an attenuation distribution image and an elasticity distribution image.

According to an aspect of the present disclosure, there is provided an ultrasound diagnostic apparatus comprising an information processing unit configured to execute: a B-mode image acquisition process of acquiring B-mode image data for a subject; an attenuation property acquisition process of acquiring attenuation property data for the subject; an attenuation distribution display process of displaying, on a display device, an attenuation distribution image in which an attenuation property map indicated by the attenuation property data is superimposed on a B-mode image indicated by the B-mode image data; a setting process of setting a region of interest on a tomographic plane from which the B-mode image data has been acquired based on a user's operation; and an elastic property acquisition process of acquiring elastic property data for the region of interest, in which the information processing unit is configured to execute the setting process when the attenuation distribution image is displayed on the display device.

In one embodiment, the information processing unit is configured to: repeatedly execute the attenuation property acquisition process and the attenuation distribution display process; and execute the setting process when the attenuation distribution image is displayed on the display device in accordance with the attenuation property acquisition process and the attenuation distribution display process that are repeatedly executed.

In one embodiment, the information processing unit is configured to execute the elastic property acquisition process based on the user's operation when the attenuation distribution image is displayed on the display device.

In one embodiment, the ultrasound diagnostic apparatus further comprises a controller configured to determine whether or not an artifact is included in a region corresponding to the region of interest in the attenuation property map, and the information processing unit is configured to execute the elastic property acquisition process when it is determined that the artifact is not included.

In one embodiment, the ultrasound diagnostic apparatus further comprises a controller configured to determine whether or not an artifact is included in the attenuation property map, and set the region of interest in a region on the tomographic plane in which the artifact is not included.

In one embodiment, the setting process includes a process of displaying the attenuation distribution image and the B-mode image side by side on the display device.

In one embodiment, the information processing unit is configured to execute an elasticity distribution display process of displaying, on the display device, an elasticity distribution image in which an elastic property map indicated by the elastic property data is superimposed on the B-mode image.

According to the aspect of the present disclosure, the operation for acquiring an ultrasound image can be facilitated.

### BRIEF DESCRIPTION OF THE DRAWINGS

FIG. 1 is a diagram showing a configuration of an ultrasound diagnostic apparatus.

FIG. 2 is a diagram showing an image displayed on a display.

FIG. 3 is a state transition diagram of the ultrasound diagnostic apparatus.

FIG. 4 is a conceptual diagram showing an operation in a case in which a state transition occurs from state B or C to state D.

FIG. 5 is a state transition diagram of a process according to an application embodiment.

### DESCRIPTION OF THE PREFERRED EMBODIMENTS

An embodiment of the present disclosure will be described with reference to the accompanying drawings. The same components shown in a plurality of drawings are designated by the same reference numerals to simplify the description thereof.

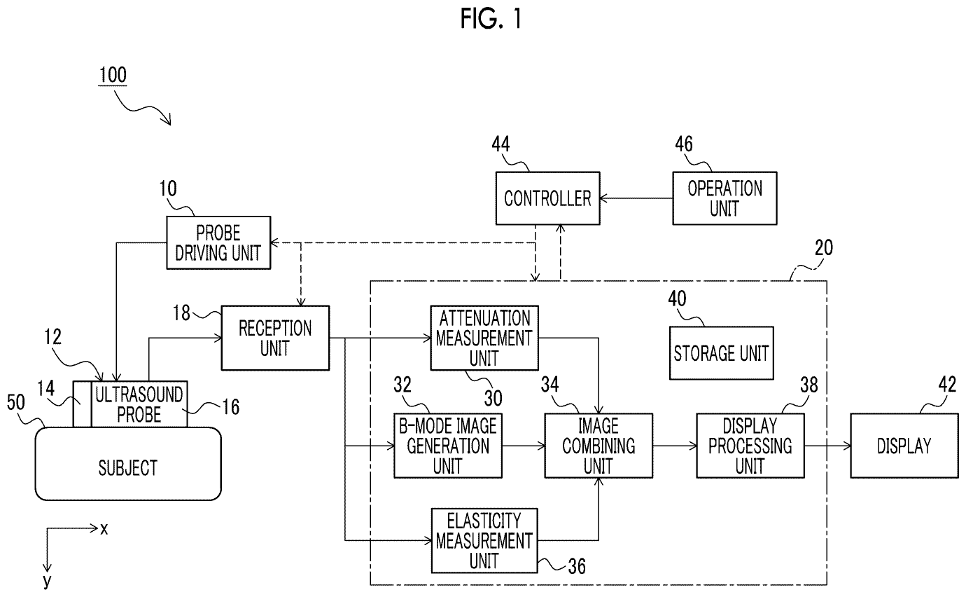

FIG. 1 shows the configuration of an ultrasound diagnostic apparatus 100 according to an embodiment of the present disclosure. The ultrasound diagnostic apparatus 100 comprises a probe driving unit 10, an ultrasound probe 12, a reception unit 18, an information processing unit 20, a display 42 (display device), a controller 44, and an operation unit 46. The operation unit 46 may comprise a button, a lever, a keyboard, a mouse, and the like. The operation unit 46 may be a touch panel provided on the display 42.

The information processing unit 20 comprises an attenuation measurement unit 30, a B-mode image generation unit 32, an image combining unit 34, an elasticity measurement unit 36, a display processing unit 38, and a storage unit 40. The information processing unit 20 and the controller 44 may be configured with, for example, one or more processors that execute a program stored in the storage unit 40. The information processing unit 20 may configure each component (the attenuation measurement unit 30, the B-mode image generation unit 32, the image combining unit 34, the elasticity measurement unit 36, and the display processing unit 38) by executing a program. Each component may read data stored in the storage unit 40, may execute an operation, and may store the data obtained as a result of the operation in the storage unit 40. The controller 44 may control the probe driving unit 10, the reception unit 18, and the information processing unit 20 according to an operation of the operation unit 46 by a user.

The ultrasound probe 12 comprises a push wave transmission unit 14 and a tracking wave transmission/reception unit 16. The probe driving unit 10 outputs a push wave drive signal and a transmission signal, respectively, as signals for generating ultrasonic waves from the push wave transmission unit 14 and the tracking wave transmission/reception unit 16. The push wave transmission unit 14 transmits a push wave to a subject 50 according to the push wave drive signal output from the probe driving unit 10, and excites a shear wave in the subject 50. The push wave transmission unit 14 may converge the push wave to a focal point determined in the subject 50.

The tracking wave transmission/reception unit 16 transmits a tracking wave as ultrasonic waves for observing a state of a biological tissue of the subject 50 or a propagation state of the shear wave based on the transmission signal output from the probe driving unit 10. The tracking wave transmission/reception unit 16 receives reflected ultrasonic waves generated by being reflected within the subject 50.

The tracking wave transmission/reception unit 16 comprises a plurality of ultrasound oscillators. The probe driving unit 10 may adjust a delay time of the transmission signal to be output to each ultrasound oscillator, and may transmit plane waves as the tracking waves from the plurality of ultrasound oscillators to the subject 50. Each ultrasound oscillator receives the reflected ultrasonic waves generated by being reflected within the subject 50, converts the reflected ultrasonic waves into a reception signal which is an electric signal, and outputs the reception signal to the reception unit 18.

The reception unit 18 performs a combining process such as phasing and addition on the reception signal output from each ultrasound oscillator to generate a plurality of pieces of y-axis direction received beam data. These plurality of pieces of y-axis direction received beam data correspond to a plurality of received beams arranged in an x-axis direction toward a depth direction (y-axis direction) of the subject 50. The reception unit 18 sequentially generates frame data over time based on a plurality of pieces of y-axis direction received beam data. That is, the reception unit 18 generates frame data arranged on a time axis based on the plurality of pieces of y-axis direction received beam data, and outputs the frame data to the attenuation measurement unit 30, the B-mode image generation unit 32, and the elasticity measurement unit 36.

The B-mode image generation unit 32 generates B-mode image data arranged on the time axis based on the frame data arranged on the time axis, and outputs the B-mode image data to the image combining unit 34. The B-mode image data is data indicating an echo image in a tomographic plane into which the plane wave transmitted from the tracking wave transmission/reception unit 16 propagates.

The image combining unit 34 outputs the B-mode image data to the display processing unit 38. The display processing unit 38 converts the B-mode image data into a video signal, and outputs the video signal to the display 42. The display 42 displays a B-mode image based on the video signal.

The attenuation measurement unit 30 obtains attenuation property data in a predetermined attenuation property region of interest on a tomographic plane based on the frame data. An attenuation property map indicated by the attenuation property data represents a distribution of attenuation rates in the attenuation property region of interest. The unit of the attenuation rate is, for example, dB/m/Hz. The attenuation property map may be a color-only image that represents the distribution of attenuation rates over the attenuation property region of interest, for example, by assigning colors with shorter wavelengths to regions with smaller attenuation rates and colors with longer wavelengths to regions with larger attenuation rates.

The attenuation property region of interest may be set in advance on the tomographic plane from which the B-mode image data has been acquired, based on the user's operation on the operation unit 46. For example, in a state in which a B-mode image is displayed on the display 42, a setting frame that specifies the attenuation property region of interest is drawn on the B-mode image displayed on the display 42 in response to an operation on the operation unit 46. The controller 44 sets the attenuation property region of interest on the tomographic plane by recognizing the setting frame drawn on the B-mode image.

The image combining unit 34 generates attenuation distribution image data showing an attenuation distribution image in which the attenuation property map is superimposed on the B-mode image based on the B-mode image data and the attenuation property data, and outputs the attenuation distribution image data to the image combining unit 34. The image combining unit 34 outputs the attenuation distribution image data to the display processing unit 38. The display processing unit 38 converts the attenuation distribution image data into a video signal, and outputs the video signal to the display 42. The display 42 displays an attenuation distribution image based on the video signal.

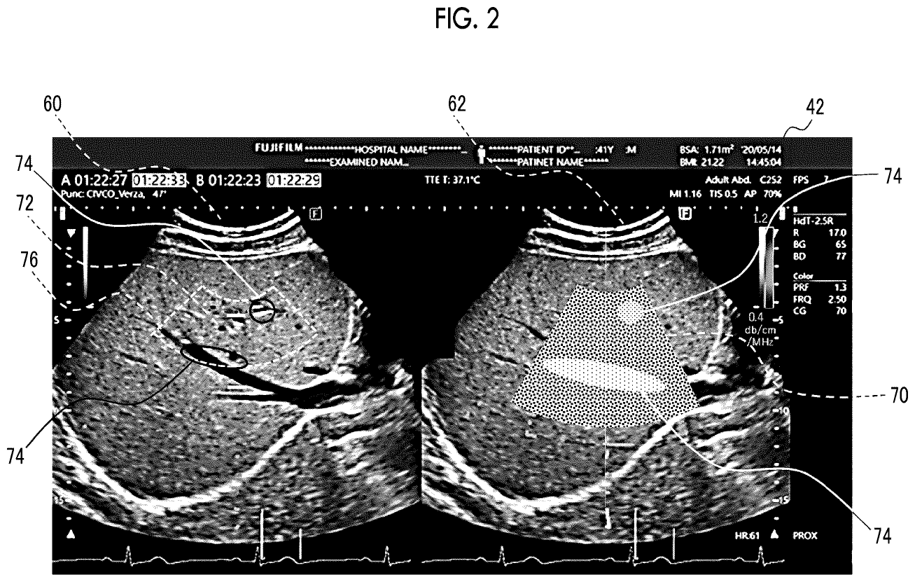

For example, as shown in FIG. 2, the display processing unit 38 may cause the display 42 to display a B-mode image 60 and an attenuation distribution image 62 side by side. The filled-in region in the attenuation distribution image 62 is an attenuation property region of interest 70.

An elastic property region of interest 72 is set on the B-mode image 60 in response to a user's operation on the operation unit 46. For example, in a state in which an attenuation property image is displayed on the display 42, a setting frame 76 that specifies the elastic property region of interest 72 is drawn on the B-mode image 60 displayed on the display 42 in response to an operation on the operation unit 46 and processing by the display processing unit 38. In FIG. 2, this setting frame 76 is indicated by a broken line. The controller 44 sets the elastic property region of interest 72 by recognizing the setting frame 76 drawn on the B-mode image. The elastic property region of interest 72 may be set to overlap the attenuation property region of interest 70 on the tomographic plane, or may be set in a region adjacent to the attenuation property region of interest 70.

Artifacts 74 appear more prominently in the attenuation distribution image 62 than in the B-mode image. Here, the artifact refers to noise that does not represent the characteristics of the subject 50. The artifacts 74 are generated by multiple reflections of the ultrasonic wave or the like due to the body surface fat, the tissue surface, or the like of the subject 50 having a property of easily reflecting the ultrasonic wave.

The elasticity measurement unit 36 measures the elastic property in the elastic property region of interest 72 by, for example, the following process. That is, the elasticity measurement unit 36 obtains an elastic modulus distribution in the elastic property region of interest 72 by the following processes (i) to (iii). The unit of the elastic modulus is, for example, N/m2.
(i) For each of a plurality of pieces of frame data arranged on the time axis, a shear wave is detected in the elastic property region of interest 72.
(ii) The time required for the shear wave to propagate a certain distance is obtained for each measurement point on the elastic property region of interest 72, thereby obtaining a distribution of propagation velocity of the shear wave in the elastic property region of interest 72.
(iii) The elastic modulus distribution in the elastic property region of interest 72 is obtained based on the distribution of the propagation velocity of the shear wave in the elastic property region of interest 72. The elastic modulus may be defined as a value proportional to a value obtained by multiplying the density of the tissue of the subject 50 by a square of the propagation velocity of the shear wave.

Specifically, the elasticity measurement unit 36 may obtain a y-axis direction propagation velocity distribution of the shear wave in the elastic property region of interest 72 by the following process described in JP2015-198843A. That is, the elasticity measurement unit 36 obtains the displacement in the y-axis direction per time 8 based on two pieces of frame data adjacent to each other on the time axis at a time interval 8, and obtains y-axis direction velocity components Vy (x, y) and Vy (x, y+Δ) of the vibration due to the shear wave. Here, Vy (x, y+Δ) indicates the y-axis direction velocity component at a position separated from the point (x, y) by Δ in the y-axis direction.

The elasticity measurement unit 36 obtains time waveforms of the y-axis direction velocity components Vy (x, y) and Vy (x, y+A). Further, the elasticity measurement unit 36 obtains a y-axis direction propagation velocity of the shear wave at the measurement point P(x, y) based on the time waveform of the y-axis direction velocity component Vy (x, y) and a movement amount on the time axis of the time waveform of the y-axis direction velocity component Vy (x, y+Δ), and obtains the y-axis direction propagation velocity distribution of the shear wave on the elastic property region of interest 72. The elasticity measurement unit 36 obtains the distribution of the elastic modulus in the elastic property region of interest 72 based on the y-axis direction propagation velocity distribution of the shear wave in the elastic property region of interest 72.

The elasticity measurement unit 36 may obtain the elastic modulus distribution based on other general processing following the above procedures (i) to (iii).

The elasticity measurement unit 36 generates elastic property data indicating an elastic property map based on the elastic modulus distribution, and outputs the elastic property data to the image combining unit 34. The elastic property map may be a color-only image that represents the distribution of elastic moduli on a plane, for example, by assigning colors with shorter wavelengths to regions with smaller elastic moduli and colors with longer wavelengths to regions with larger elastic moduli.

The image combining unit 34 generates elasticity distribution image data showing an elasticity distribution image in which an elastic property map is superimposed on a B-mode image, based on the elastic property data and the B-mode image data. The image may be an image obtained by subjecting a B-mode image to the color corresponding to the elastic modulus. The image combining unit 34 outputs the elasticity distribution image data to the display processing unit 38. The display processing unit 38 converts the elasticity distribution image data into a video signal, and outputs the video signal to the display 42. The display 42 displays an elasticity distribution image based on the video signal.

The ultrasound diagnostic apparatus 100 operates in any one of the operation modes of the B-mode, the attenuation measurement mode, and the elasticity measurement mode under the control of the controller 44 in accordance with the setting operation of the operation mode in the operation unit 46. The B-mode is an operation mode in which a B-mode image is displayed based on the B-mode image data generated by the B-mode image generation unit 32. The attenuation measurement mode is an operation mode in which an attenuation distribution image is displayed based on the attenuation distribution image data generated by the B-mode image generation unit 32, the attenuation measurement unit 30, and the image combining unit 34. The elasticity measurement mode is an operation mode in which an elasticity distribution image is displayed based on the elasticity distribution image data generated by the B-mode image generation unit 32, the elasticity measurement unit 36, and the image combining unit 34.

In the following description, the attenuation measurement mode is sometimes referred to as an ATT mode (ATTenuation measurement), and the elasticity measurement mode is sometimes referred to as an SWE mode (Shear Wave Elastography). Only one of an operation in the B-mode, an operation in the ATT mode, or an operation in the SWE mode may be executed, or two or three of these three operation modes may be executed in a time-division manner.

The image combining unit 34 may output one or two of the B-mode image data, the attenuation distribution image data, or the elasticity distribution image data to the display processing unit 38 in accordance with the set operation mode, and display one or two of the B-mode image, the attenuation distribution image, or the elasticity distribution image on the display 42. In addition, the image combining unit 34 may output the B-mode image data, the attenuation distribution image data, and the elasticity distribution image data to the display processing unit 38 in accordance with the user's operation of the operation unit 46, and display the B-mode image, the attenuation distribution image and the elasticity distribution image on the display 42.

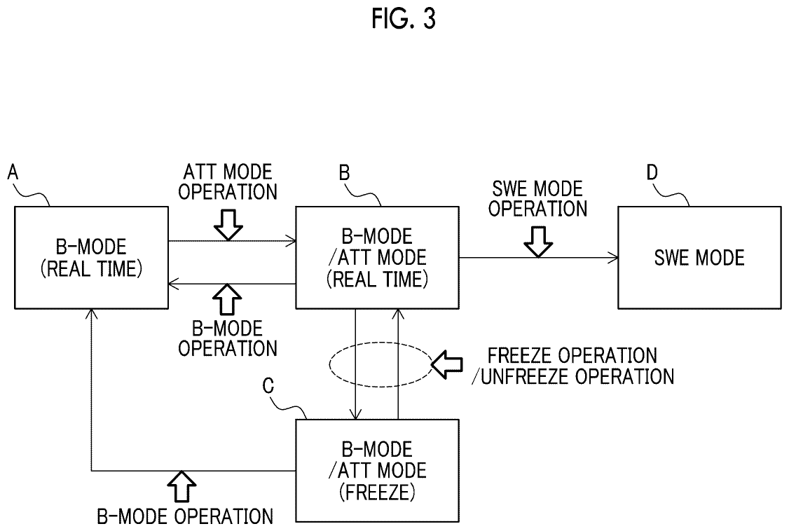

FIG. 3 shows a state transition diagram of the ultrasound diagnostic apparatus 100. State A is a state in which the ultrasound diagnostic apparatus operates in the B-mode and displays only a B-mode image in real time. Here, displaying a B-mode image in real time means that the displayed B-mode image is updated sequentially over time based on B-mode image data that is generated sequentially over time.

State B is a state in which the operation in the B-mode and the operation in the ATT mode are executed in a time-division manner, and a B-mode image and an attenuation distribution image are displayed in real time. In real-time display of an attenuation distribution image, both the attenuation property map and the B-mode image constituting the attenuation distribution image are updated sequentially over time. In state B, a B-mode image 60 and an attenuation distribution image 62 may be displayed side by side, as shown in FIG. 2.

State C is a state in which the operation in the B-mode and the operation in the ATT mode are executed in a time-division manner, and the B-mode image and the attenuation distribution image are displayed in a frozen state. Here, displaying an image in a frozen state means displaying one frame of an image as a still image without updating it. In state C, similarly to state B, a B-mode image 60 and an attenuation distribution image 62 may be displayed side by side, as shown in FIG. 2.

State D is a state in which the operation in the SWE mode is executed and an elasticity distribution image is displayed. In state D, an elasticity distribution image may be displayed instead of the B-mode image 60 shown in FIG. 2. That is, an elasticity distribution image in which an elastic property map is shown in the elastic property region of interest 72 set by the setting frame 76 may be displayed side by side with the attenuation distribution image 62. While the elastic property map constituting the elasticity distribution image is updated sequentially over time, the B-mode image on which the elasticity distribution image is based may be a still image that is not updated.

An example of the operation of the ultrasound diagnostic apparatus 100 will be described with reference to FIG. 3. The state of the ultrasound diagnostic apparatus 100 is initially set to state A, and the ultrasound diagnostic apparatus 100 operates in the B-mode. In a case in which the user performs an ATT mode operation on the operation unit 46 to operate in the ATT mode, the state of the ultrasound diagnostic apparatus 100 transitions from state A to state B, and the ultrasound diagnostic apparatus 100 executes the operation in the ATT mode in addition to the operation in the B-mode.

When the state of the ultrasound diagnostic apparatus 100 is state B, a freeze operation for freezing and displaying the B-mode image is performed by the user on the operation unit 46, whereby the state of the ultrasound diagnostic apparatus 100 transitions from state B to state C. This allows the ultrasound diagnostic apparatus 100 to freeze and display the B-mode image and the attenuation distribution image. When the state of the ultrasound diagnostic apparatus 100 is state C, an unfreeze operation for unfreezing the B-mode image and the attenuation distribution image is performed by the user on the operation unit 46, whereby the state of the ultrasound diagnostic apparatus 100 transitions from state C to state B. This allows the ultrasound diagnostic apparatus 100 to display the B-mode image and the attenuation distribution image in real time.

When the state of the ultrasound diagnostic apparatus 100 is state B or C, a B-mode operation to start the operation in the B-mode only is performed by the user on the operation unit 46, whereby the state of the ultrasound diagnostic apparatus 100 returns to state A. This causes the ultrasound diagnostic apparatus 100 to execute the operation in the B-mode.

In a case in which the state of the ultrasound diagnostic apparatus 100 is state B, and as shown in FIG. 2, and an attenuation distribution image 62 and a B-mode image 60 are displayed on the display 42, the user may set an elastic property region of interest 72 by drawing a setting frame 76 on the B-mode image 60 while referring to the attenuation distribution image 62.

In general, in a case in which no artifacts appear in an attenuation distribution image, artifacts often also do not appear in an elasticity distribution image in the region from which the attenuation distribution image was acquired. Therefore, the user may, for example, confirm that no artifacts appear in the attenuation distribution image, and set a region in which no artifacts appear in the attenuation distribution image as the elastic property region of interest.

When the state of the ultrasound diagnostic apparatus 100 is state B, an SWE mode operation to start the operation in the SWE mode is performed by the user on the operation unit 46, whereby the state of the ultrasound diagnostic apparatus 100 transitions to state D. This causes the ultrasound diagnostic apparatus 100 to execute the operation in the SWE mode. By executing the operation in the SWE mode, the ultrasound diagnostic apparatus 100 displays an elasticity distribution image. In the SWE mode, the B-mode images constituting the elasticity distribution image may be displayed in a frozen state.

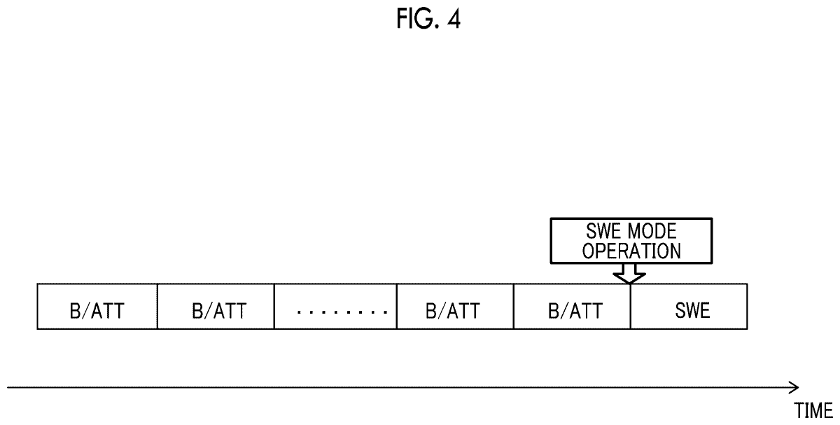

FIG. 4 shows a conceptual diagram of the operation in a case in which the state of ultrasound diagnostic apparatus 100 transitions to state D as a result of an SWE mode operation being performed by the user on the operation unit 46 when the state of ultrasound diagnostic apparatus 100 is state B. In FIG. 4, the time slots denoted by “B/ATT” indicate time slots during which the ultrasound diagnostic apparatus 100 is operating in the B-mode and the ATT mode. The time slot denoted by “SWE” indicates a time slot during which the ultrasound diagnostic apparatus 100 is operating in the SWE mode. While the B-mode and the ATT mode are being repeatedly executed, the user refers to the attenuation distribution image displayed on the display 42 and sets, for example, a region where no artifacts occur as an elastic property region of interest. When the setting of the elastic property region of interest is completed, a SWE mode operation is performed on the operation unit 46, whereby the ultrasound diagnostic apparatus 100 operates in the SWE mode, generates elasticity distribution image data, and displays the elasticity distribution image on the display 42.

The state transitions shown in FIG. 3 are implemented by the information processing unit 20 executing the following processes. That is, the information processing unit 20 executes a B-mode image acquisition process, an attenuation property acquisition process, an attenuation distribution display process, a setting process, an elastic property acquisition process, and an elasticity distribution display process.

The B-mode image acquisition process is a process of acquiring B-mode image data for the subject 50. The attenuation property acquisition process is a process of acquiring attenuation property data for the subject 50. The attenuation distribution display process is a process of displaying, on the display 42 (display device), an attenuation distribution image in which an attenuation property map indicated by the attenuation property data is superimposed on a B-mode image indicated by the B-mode image data. The setting process is a process of setting an elastic property region of interest (region of interest) on a tomographic plane from which the B-mode image data has been acquired based on a user's operation. The elastic property acquisition process is a process of acquiring elastic property data for the elastic property region of interest. The information processing unit 20 executes a setting process when the attenuation distribution image is displayed on the display 42. The elasticity distribution display process is a process of displaying, on the display 42, an elasticity distribution image in which an elastic property map indicated by the elastic property data is superimposed on a B-mode image.

In the process shown in FIG. 4, the information processing unit 20 repeatedly executes the attenuation property acquisition process and the attenuation distribution display process. The information processing unit 20 executes the setting process when the attenuation distribution image is displayed on the display 42 in accordance with the attenuation property acquisition process and the attenuation distribution display process that are repeatedly executed.

In the configuration and processing of the ultrasound diagnostic apparatus 100, the user may refer to the attenuation distribution image displayed on the display 42 and set a region where no artifacts occur as an elastic property region of interest. The frame rate when generating attenuation distribution image data in the B-mode and the ATT mode is greater than the frame rate when generating elasticity distribution image data in the SWE mode. The reason for this is that in the SWE mode, in order to suppress heat generation in the push wave transmission unit 14, the interval at which the push wave is repeatedly transmitted from the push wave transmission unit 14 needs to be sufficiently long. Therefore, according to the processing of the present embodiment, the operation of setting the elastic property region of interest is performed easily and quickly compared to the case in which the elastic property region of interest is set by trial and error while avoiding artifacts in the operation in the SWE mode.

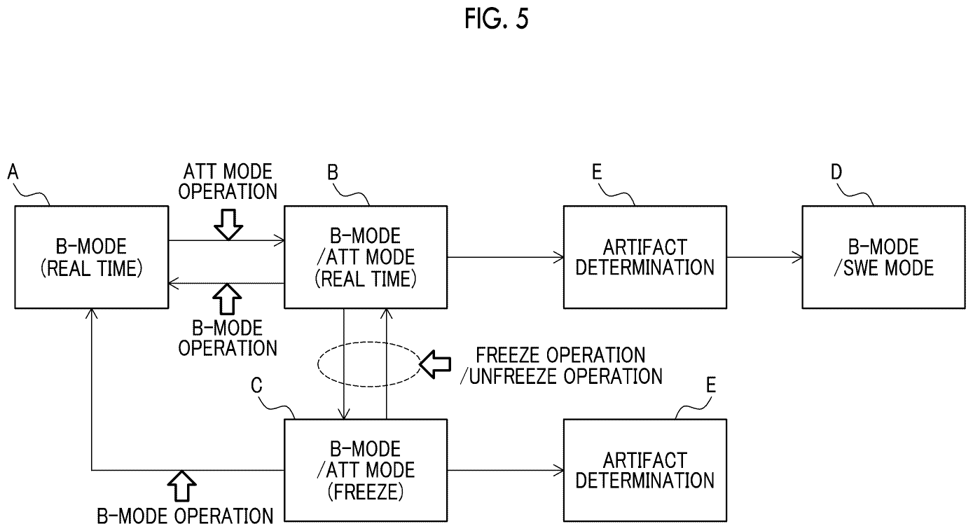

FIG. 5 shows a state transition diagram of a process according to an application embodiment of the present disclosure. In this process, when the state of the ultrasound diagnostic apparatus 100 is state B, the controller 44 executes artifact determination E on the attenuation distribution image data. The artifact determination E is a process of determining whether or not an artifact has occurred in a determination region in which an elastic property region of interest has been set, among the regions of the attenuation distribution image indicated by the attenuation distribution image data. This determination may be made based on the presence or absence of an image of a blood vessel in the determination region, the presence or absence of an image of multiple reflections of ultrasonic waves, unevenness of attenuation distribution, and the like. The unevenness of the attenuation distribution may be expressed by the degree of variation in the attenuation rate, such as the variance or standard deviation of the attenuation rate in the determination region.

When it is determined that no artifacts have occurred in the determination region, the controller 44 changes the state of the ultrasound diagnostic apparatus 100 from state B to state D, and operates the ultrasound diagnostic apparatus 100 in the SWE mode. On the other hand, when it is determined that an artifact has occurred in the determination region, the controller 44 maintains the state of the ultrasound diagnostic apparatus 100 in state B. Note that the controller 44 may execute artifact determination E when the state of the ultrasound diagnostic apparatus 100 is state C, and determine whether or not an artifact has occurred in the determination region.

In artifact determination E, the controller 44 may execute a process of setting an elastic property region of interest in a region of the attenuation distribution image where no artifacts occur. In addition, the controller 44 may execute a process of changing the range of the elastic property region of interest set by operating the operation unit 46 such that the elastic property region of interest is set in a region where no artifacts occur.

In this manner, the controller 44 determines whether or not an artifact is included in the region in the attenuation property map that corresponds to the elastic property region of interest. The elasticity measurement unit 36 in the information processing unit 20 executes an elastic property acquisition process when it is determined that no artifacts are included. In addition, the controller 44 may determine whether or not an artifact is included in the attenuation property map, and set the elastic property region of interest in a region on the tomographic plane in which the artifact is not included.

According to such a process, the controller 44 determines whether or not an artifact has occurred. In an application embodiment, the controller 44 sets the elastic property region of interest to a region on the tomographic plane in which the artifact is not included. This reduces the operational burden on the user to cause the ultrasound diagnostic apparatus 100 to acquire elasticity distribution image data.

### Configuration of Present Disclosure

### Configuration 1:

An ultrasound diagnostic apparatus comprising an information processing unit configured to execute:
a B-mode image acquisition process of acquiring B-mode image data for a subject;
an attenuation property acquisition process of acquiring attenuation property data for the subject;
an attenuation distribution display process of displaying, on a display device, an attenuation distribution image in which an attenuation property map indicated by the attenuation property data is superimposed on a B-mode image indicated by the B-mode image data;
a setting process of setting a region of interest on a tomographic plane from which the B-mode image data has been acquired based on a user's operation; and
an elastic property acquisition process of acquiring elastic property data for the region of interest,
in which the information processing unit is configured to execute the setting process when the attenuation distribution image is displayed on the display device.

### Configuration 2:

The ultrasound diagnostic apparatus according to Configuration 1,
in which the information processing unit is configured to:
   repeatedly execute the attenuation property acquisition process and the attenuation distribution display process; and
   execute the setting process when the attenuation distribution image is displayed on the display device in accordance with the attenuation property acquisition process and the attenuation distribution display process that are repeatedly executed.

### Configuration 3:

The ultrasound diagnostic apparatus according to Configuration 1 or 2,
in which the information processing unit is configured to execute the elastic property acquisition process based on the user's operation when the attenuation distribution image is displayed on the display device.

### Configuration 4:

The ultrasound diagnostic apparatus according to any one of Configurations 1 to 3, further comprising a controller configured to determine whether or not an artifact is included in a region corresponding to the region of interest in the attenuation property map,
in which the information processing unit is configured to execute the elastic property acquisition process when it is determined that the artifact is not included.

### Configuration 5:

The ultrasound diagnostic apparatus according to any one of Configurations 1 to 3, further comprising a controller configured to determine whether or not an artifact is included in the attenuation property map, and set the region of interest in a region on the tomographic plane in which the artifact is not included.

### Configuration 6:

The ultrasound diagnostic apparatus according to any one of Configurations 1 to 5, in which the setting process includes a process of displaying the attenuation distribution image and the B-mode image side by side on the display device.

### Configuration 7:

The ultrasound diagnostic apparatus according to any one of Configurations 1 to 5, in which the information processing unit is configured to execute an elasticity distribution display process of displaying, on the display device, an elasticity distribution image in which an elastic property map indicated by the elastic property data is superimposed on the B-mode image.

What is claimed is:

1. An ultrasound diagnostic apparatus comprising an information processing unit configured to execute:
a B-mode image acquisition process of acquiring B-mode image data for a subject;
an attenuation property acquisition process of acquiring attenuation property data for the subject;
an attenuation distribution display process of displaying, on a display device, an attenuation distribution image in which an attenuation property map indicated by the attenuation property data is superimposed on a B-mode image indicated by the B-mode image data;
a setting process of setting a region of interest on a tomographic plane from which the B-mode image data has been acquired based on a user's operation; and
an elastic property acquisition process of acquiring elastic property data for the region of interest,
wherein the information processing unit is configured to execute the setting process when the attenuation distribution image is displayed on the display device.

2. The ultrasound diagnostic apparatus according to claim 1,
wherein the information processing unit is configured to:
repeatedly execute the attenuation property acquisition process and the attenuation distribution display process; and
execute the setting process when the attenuation distribution image is displayed on the display device in accordance with the attenuation property acquisition process and the attenuation distribution display process that are repeatedly executed.

3. The ultrasound diagnostic apparatus according to claim 1,
wherein the information processing unit is configured to execute the elastic property acquisition process based on the user's operation when the attenuation distribution image is displayed on the display device.

4. The ultrasound diagnostic apparatus according to claim 1, further comprising a controller configured to determine whether or not an artifact is included in a region corresponding to the region of interest in the attenuation property map,
wherein the information processing unit is configured to execute the elastic property acquisition process when it is determined that the artifact is not included.

5. The ultrasound diagnostic apparatus according to claim 1, further comprising a controller configured to determine whether or not an artifact is included in the attenuation property map, and set the region of interest in a region on the tomographic plane in which the artifact is not included.

6. The ultrasound diagnostic apparatus according to claim 1,
wherein the setting process includes a process of displaying the attenuation distribution image and the B-mode image side by side on the display device.

7. The ultrasound diagnostic apparatus according to claim 1,
wherein the information processing unit is configured to execute an elasticity distribution display process of displaying, on the display device, an elasticity distribution image in which an elastic property map indicated by the elastic property data is superimposed on the B-mode image.
